(19) United States
(12) Patent Application Publication
(10) Pub. No.: US 2010/0143447 A1
(43) Pub. Date: Jun. 10, 2010

(54) WOUND OR TISSUE DRESSING COMPRISING LACTIC ACID BACTERIA

(75) Inventors: John E. Hansen, Soborg (DK); Lene Karin Jespersen, Fredensborg (DK)

Correspondence Address: KING & SPALDING, 1185 AVENUE OF THE AMERICAS, NEW YORK, NY, 10036-4003, US

(73) Assignee: FERROSAN A/S, Søborg (DK)

(21) Appl. No.: 12/519,548

(22) PCT Filed: Dec. 19, 2007
(86) PCT No.: PCT/DK2007/050196

§ 371 Date: Jan. 21, 2010

(30) Foreign Application Priority Data
Dec. 19, 2006 (DK) PA 2006 01667

(51) Int. Cl. A61L 15/36; A61K 35/74; A61P 17/02

(52) U.S. Cl. 424/445; 424/93.44; 424/93.45; 424/93.4

(57) ABSTRACT

The present invention is directed to a wound or tissue dressing comprising a bacteria having the property of producing lactic acid from sugars by fermentation of the sugars. The bacteria preferably belongs to the family of lactic acid bacteria. The family of lactic acid bacteria refers to any bacteria belonging to a genus selected from the group consisting of Carnobacterium, Enterococcus, Lactobacillus, Lactococcus, Leuconostoc, Oenococcus, Pediococcus, Streptococcus, Tetragenococcus, Vagococcus and Weissella. There is also provided a wound or tissue dressing comprising an absorbent compound for absorbing wound exudate, wherein said wound or tissue dressing is attached to or comprises a lactic acid bacterium. The utility of the present invention is demonstrated by use of the wound or tissue dressings in methods for treating a wound or damaged tissue in an individual, said method comprising the steps of contacting said wound or damaged tissue with the wound or tissue dressing according to the invention, thereby treating the wound or damaged tissue. The treatment results in healing of the wound or in accelerated healing of the wound. There is also provided the use of a lactic acid bacteria in the manufacture of a wound or tissue dressing for treating or accelerating the healing of a wound in an individual.

This patent application is a non-provisional patent application and claims the benefit of U.S. 60/870,625 filed on Dec. 19, 2006, which is hereby incorporated by reference in its entirety. All patent and non-patent references cited in U.S. 60/870,625 as well as in the present application, are also incorporated herein by reference.

### FIELD OF INVENTION

The present invention relates to a wound or tissue dressing comprising lactic acid producing bacteria. Also provided are uses of such a wound or tissue dressing in healing wounds or in accelerating the healing of wounds.

### BACKGROUND OF INVENTION

Wounds to the skin and the underlying tissues of animals may be caused by e.g. friction, abrasion, laceration, pressure, burning, or chemical irritation. Tissue damage may also result from internal metabolic or physical dysfunction, including, but not limited to, bone protrudence, diabetes, circulatory insufficiencies, or inflammatory processes.

Wounds can be classified as acute or chronic. Wounds that are not progressing in 4 weeks and not healed within 8 weeks of formation are classified chronic. Chronic wounds are often found to be infected and the wound fluid in chronic wounds have been shown to have a higher pH and a higher level of protease activity than wound fluid from acute wounds. The higher level of proteases in chronic wounds can either origin from cells in the tissue or from bacteria in the wound exudate.

A wound to the skin and/or damage to the underlaying tissues significantly reduce the protective function of the skin. Consequently, damaged skin results in an increased risk of infection of the under laying tissue by infectious agents such as bacteria and vira.

Areas of damaged skin are conventionally protected by the application of a wound or tissue dressing which facilitates wound healing. Wound or tissue dressings generally provide a suitable environment for wound healing by absorbing exudate, keeping the wound bed moist , and by protecting the wound and new tissue growth from bacterial contamination.

Despite careful attention by medical personal in hospitals and health clinics, many wounds are often infected during the wound healing process—either in the health care environment or when out-patients are undergoing recovery outside the health care environment.

Several types of wound or tissue dressings exist. Most wound or tissue dressings are designed to maintain a moist wound bed. The most commonly used wound or tissue dressing are briefly introduced below.

Gauze dressings are dressing made of cotton, available in a number of forms including sponges, pads, ropes, strips, and rolls, gauze can also be impregnated with petroleum, antimicrobials, and saline. With removal of a dried gauze dressing, there is a risk of wound damage to the healing skin surrounding the wound.

Hydrocolloid dressings are among the oldest and most frequently used wound or tissue dressings. Hydrocolloids are either occlusive (i.e. they do not allow air to escape through the dressing), or semi-occlusive (i.e. they do allow some air to escape through the dressing) and they are designed to seal the wound bed to retain and interact with exudate to promote healing. They used on dry wounds with necrotic tissue in order to get autolytic debridement and also as a protective layer on newly formed epithelial layer.

Hydrogel dressings are either sheets of cross-linked polymers or hydrogel impregnated gauze, or non-woven sponge, used to cover a wound. The hydrogel dressing can be in the form of a hydrogel sheet dressing or in the form of an amorphous hydrogel dressing. Hydrogel sheet dressings are indicated for, wounds with necrosis or slough, and burns. An amorphous hydrogel dressing is a soft, formless gel comprised of either polymers or copolymers and up to 95 percent water, whereas a hydrogel sheet dressing is a firm sheet. Amorphous hydrogels carry the same indications as hydrogel sheets and they can also be used to lightly pack full-thickness wounds.

Alginate dressings are highly absorbent, biodegradable dressings derived from seaweed. They are used for wounds with moderate to large amounts of exudate, and for wounds requiring packing. These dressings work by combining with the wound exudate to form a hydrophylic gel that creates a moist healing environment.

Foam dressings are semipermeable sheets of a polymer, such as polyurethane, that provide a specific, controlled moisture and temperature environment for wound healing. They are indicated for wounds with moderate to heavy exudate. Foam dressings are non-adherent and can repel contaminants.

Transparent film dressings are made of e.g. polyurethane, polyamide, polyurethane or gelatin. Although they are waterproof, transparent film dressings are somewhat porous allowing for oxygen and moisture to cross through their barriers. They are non-absorptive so they must be changed often for wounds with exudate. They are generally effective on dry wounds with necrotic tissue in need of autolytic debridement. Transparent film dressings are also used as a secondary material to secure e.g. non-adhesive gauzes and other types of dry dressings.

In addition to dressings meant to keep the wound bed moist, there are also dressings that act as a substrate for proteases and/or scaffolds for new tissue. These dressings can be based on collagen or gelatine.

Halper et al. (2003) have disclosed wound healing and angiogenic properties of supernatants from Lactobacillus cultures (Exp. Biol. and Med., vol. 228, pp. 1329-1337.

EP 568 334 is directed to a collagen-containing sponge comprising an absorbable gelatin sponge, collagen, and an active ingredient. The absorbable gelatin sponge can be crosslinked.

U.S. Pat. No. 5,399,361 is directed to a collagen-containing sponge comprising an absorbable, cross-linked gelatin sponge, soluble collagen, and a therapeutically effective amount of an active ingredient.

EP 1 082 964 is directed to a lactic acid bacteria-containing composition comprising lactic acid bacteria showing pharmacologic action in the digestive tract and/or the urogenital organ, wherein the adhesion of said lactic acid bacteria to the digestive tract and/or the urogenital organ is enhanced to potentiate said pharmacologic action.

U.S. Pat. No. 6,716,435 and U.S. Pat. No. 7,025,974 disclose an absorbent product comprising a liquid absorbent structure and a viable Bacillus coagulans. The Bacillus coagulans is present on an external surface of said product.

### SUMMARY OF INVENTION

The present invention in one aspect is directed to a wound or tissue dressing comprising lactic acid producing bacteria. Further metabolites, in addition to lactic acid, are preferably also produced by said lactic acid producing bacteria. Examples of such metabolites are e.g. one or both of diacetyl and hydrogen peroxide. The lactic acid producing bacteria can further produce e.g. extracellular proteases and bacteriocins which have beneficial effects on the wound healing process.

The wound or tissue dressing according to the present invention is capable of promoting wound healing in an individual in need thereof. The individual is preferably an animal, such as a human being.

Wound healing is a complex regeneration process, which is characterised by intercalating degradation and re-assembly of connective tissue and epidermal layer.

One reason for wounds not to heal can be infection with putrefactive bacteria causing an unbalanced wound environment. Most bacteria have a growth optimum at pH between 6.5 and 7.5. It is believed that a lowering of the pH of the wound environment will reduce the number and/or growth of putrefactive bacteria to the extent that the reduction positively correlates with an improved wound healing.

The effect exerted by the dressing according to the present invention is believed to be also correlated with the fact that lactid acid producing bacteria present in the dressing express certain desirable bacteriocins in the form of toxins that inhibit undesirable bacterial growth.

By lowering the pH in the wound environment, it is believed that one will also reduce the activity of proteases present in the wound. Especially in chronic wounds are protease activities believed to be out of balance.

While it is true that proteases are needed for the wound healing process, high levels of protease activities are likely to maintain the wound in a chronic state. Most proteases have a pH optimum at alkaline pH and a weakly acidic environment may therefore promote healing of open wounds by inhibiting the action of undesirable proteases. By operably contacting the chronic wound with a bacteria capable of producing certain anti-bacterial and pH lowering components, putrefactive bacteria can be neutralised and/or eliminated and a weakly acidic wound environment can be obtained and sustained over periods of time relevant for effectively promoting wound healing.

Accordingly, in one embodiment there is provided a wound or tissue dressing comprising a non-pathogenic bacteria capable of producing lactic acid by fermentation of polysaccharides or residues thereof. The bacteria can be spore forming—or the bacteria can be incapable of forming spores as the case may be. In one preferred embodiment, the lactic acid producing bacteria are not spore-forming bacteria.

The bacteria preferably belongs to the family of lactic acid producing bacteria, more preferably to a family of lactic acid producing bacteria capable of producing further metabolites, in addition to lactic acid. Examples of such further metabolites are e.g. one or both of diacetyl and hydrogen peroxide. The family of lactic acid producing bacteria is preferably one also capable of producing e.g. extracellular proteases and bacteriocins which have beneficial effects on the wound healing process.

Preferred lactic acid producing bacteria according to the present invention are preferably capable of one or more of 1) lowering the pH in an open wound environment, 2) securing an intraspecies competitive exclusion, i.e. preventing growth of undesirable bacterial species, 3) exerting an immunomodulatory effect, and 4) production of certain bacteriocins, such as toxins capable of sustaining a wound healing process.

The family of lactic acid bacteria according to the present invention preferably refers to any bacteria belonging to a genus selected from the group consisting of Carnobacterium, Enterococcus, Lactobacillus, Lactococcus, Leuconostoc, Oenococcus, Pediococcus, Streptococcus, Tetragenococcus, Vagococcus and Weissella. In one embodiment, the lactic acid bacteria employed do not belong to the genus Bacillus, including the species Bacillus coagulans.

The dressing according to the present invention can be regarded as “sterile”—apart from the fact that the dressing comprises lactic acid producing bacteria. Accordingly, the term “sterile” will be used herein to characterise the dressing as being free from viable microorganisms (i.e. “sterile”) apart from the optionally freeze dried and/or encapsulated lactic acid bacteria forming part of the dressings.

The dressing according to the invention comprises an absorbant compound. The absorbant compound can comprise the lactic acid producing bacteria, or the lactic acid producing bacteria can be present in the dressing in a compartment different from the absorbant compound. The lactic acid producing bacteria can be present on the surface part of the absorbant compound or the lactic acid producing bacteria can be present internally in the absorbant compound—in principle either by physical entrapment, by chemical bonding, or otherwise.

When being initially present in a compartment different from the absorbant compound, i.e. prior to the dressing contacting a wound or tissue, the lactic acid producing bacteria can be present in said different compartment either by physical entrapment e.g. within a matrix present in said different compartment, by chemical bonding to such a matrix, for example a gelatin and/or collagen matrix, i.e. an open cell structure, such as e.g. a sponge, or otherwise, e.g. by encapsulation with gelatin and/or collagen, thereby e.g. forming gel-like particles incapable of diffusing out of the matrix. The gel-like particles comprising the optionally lyophilized lactic acid producing bacteria can be degraded when coming into contact with wound exudate, which degradation initially releases the lactic acid producing bacteria to the space of the different compartment, and optionally subsequently releases the lactic acid producing bacteria to the wound or to at least part of the absorbant compound.

The compartment different from the absorbant compound can be separated from the absorbant compound by a barrier or semi-permeable or permeable membrane. The barrier or membrane can allow metabolites produced by the lactic acid producing bacteria to diffuse over the membrane and e.g. into the wound area and/or the barrier or membrane can allow the lactic acid producing bacteria themselves to diffuse over the membrane and e.g. into the wound area.

The lactic acid producing bacteria are preferably lyophilized (freeze-dried) by any state-of-the-art method for lyophilizing lactic acid producing bacteria. The lactic acid bacteria in their optionally lyophilized state can further be encapsulated or contained in biodegradable microspheres, e.g. biodegradable microspheres which become degraded when contacted by wound exudate.

The absorbant compound can be in contact with one or more pad(s) or compartment(s) comprising the lactic acid bacteria in their optional lyophilized state. The pad or compartment can be positioned on the proximal side of the absorbant compound, i.e. between the absorbant compound and the wound, and/or the pad comprising the lactic acid producing bacteria in their optional lyophilized state can be positioned on the distal side of the absorbant compound (in relation to the wound or tissue to be treated).

The lactic acid producing bacteria in their optional lyophilized state can generally be contained in a pad or compartment defined by or comprising a permeable or semi-permeable membrane allowing the lactic acid producing bacteria, or their metabolites only, as the case may be, to migrate from the pad or compartment to the absorbant compound and/or to the wound environment.

The pad or compartment can also comprise different membrane portions, wherein each portion has permeable or semi-permeable characteristics with respect to the migration of the lactic acid producing bacteria and their metabolites. One area or side of the pad or compartment can e.g. comprise a membrane portion allowing migration of the lactic acid producing bacteria to the wound, whereas another side or area of the pad allows migration to the absorbant compound of metabolites only. The metabolites can be e.g. lactic acid and/or bacteriocins.

Biodegradable microspheres used for encapsulation of the lactic acid producing bacteria are disclosed herein below in more detail.

In another embodiment there is provided a wound or tissue dressing comprising an absorbent compound for absorbing wound exudate, wherein said wound or tissue dressing is attached to or comprises a lactic acid producing bacterium. The lactic acid producing bacterium is—in one embodiment—preferably present internally in the wound or tissue dressing, or internally in the absorbant compound, i.e. not on an external surface thereof.

In one embodiment, the above-cited wound or tissue dressing according to the present invention further comprises at least one adhesive surface suitable for contacting a wound and/or at least one topfilm for preventing leakage of wound exudate from the absorbant compound.

In yet another embodiment there is provided a wound or tissue dressing comprising an absorbent compound for absorbing wound exudate, wherein said absorbent compound is attached to or comprises a lactic acid producing bacterium, optionally in lyophilized and/or encapsulated form.

In yet another embodiment there is provided a dual compartment syringe optionally fitted with a plunger, wherein one compartment comprises e.g. a hydrogel as disclosed herein below in more detail (FIG. 1, panel C) and wherein the other compartment comprises lactic acid producing bacteria, optionally in lyophilized and/or encapsulated form.

In a further aspect there is provided a method for manufacturing a wound or tissue dressing comprising an absorbent compound according to the present invention, said method comprising the steps of providing a lactic acid bacteria, mixing or attaching said lactic acid bacteria with the wound or tissue dressing or with the absorbent compound of the wound or tissue dressing, thereby obtaining a wound or tissue dressing according to the present invention.

In a still further aspect there is provided a method for treating a wound in an individual, said method comprising the steps of contacting said wound with the wound or tissue dressing according to the invention, thereby treating the wound. The treatment results in healing of the wound or in accelerated healing of the wound.

There is also provided the use of a lactic acid bacteria in the manufacture of a wound or tissue dressing for treating or accelerating the healing of a wound in an individual.

In yet another aspect there is provided the use of a lactic acid bacteria in the manufacture of an absorbent compound for use in a wound or tissue dressing for treating or accelerating the healing of a wound in an individual.

### DEFINITIONS

“Wound” refers broadly to injuries to the skin and underlaying (subcutaneous) tissue initiated in different ways (e.g., pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

“Partial thickness wound” refers to wounds that encompass Grades I-III; examples of partial thickness wounds include burn wounds, pressure sores, venous stasis ulcers, and diabetic ulcers.

“Deep wound” is meant to include both Grade III and Grade IV wounds. The present invention contemplates treating all wound types, including deep wounds and chronic wounds.

“Chronic wound” refers to a wound that has not progressed within 4 weeks and/or not healed within 8 weeks.

“Positioning the wound or tissue dressing in or on the wound” means contacting some part of the wound with the wound or tissue dressing.

“Promote wound healing” and “enhance wound healing,” and similar phrases, refer to either the induction of the formation of granulation tissue, of wound contraction, and/or the induction of epithelialization (i.e., the generation of new cells in the epithelium). Wound healing is conveniently measured by decreasing wound area.

“Alginate” refers to a linear co-polymer with homopolymeric blocks of (1-4)-linked β-D-mannuronate (M) and its C-5 epimer α-L-guluronate (G) residues, respectively, covalently linked together in different sequences or blocks.

“Hydrocolloid” refers to a colloid system in which the colloid-forming components are dispersed in water, but not cross-linked. A colloid system is a system or mixture in which two substances are interspersed between each other. A hydrocolloid has colloid particles spread throughout water and depending on the quantity of water available can take on different states, e.g: gel-like consistency or a sol (liquid). Hydrocolloids can be either irreversible (single state) or reversible. Examples include carrageenan, gelatin, cellulose, and pectin.

“Hydrogel” refers to a gel wherein water is the dispersion medium and wherein the gel-forming components are cross-linked.

“Lactic acid bacteria” as used herein denotes any bacteria which has the property of producing lactic acid from sugars by fermentation. Accordingly, the taxonomy of lactic acid bacteria as used herein can be based on the Gram reaction and the production of lactic acid from various fermentable carbohydrates. “Lactic acid bacteria” includes any bacteria belonging to a genus selected from the group consisting of Carnobacterium, Enterococcus, Lactobacillus, Lactococcus, Leuconostoc, Oenococcus, Pediococcus, Streptococcus, Tetragenococcus, Vagococcus and Weissella.

Reference is made to Stiles and Holzapfel; Int. J. Food Microbiol. (1997), 36(1), pp. 1-29 and to the monograph “Genera of Lactic Acid Bacteria” by Holzapfel and Wood, published in 1998 as vol. 2 by Springer Verlag in the series “The Lactic Acid Bacteria” (ISBN: 0-7514-0215-X).

“Wound healing-promoting substance” is any substance capable of accelerating the wound healing process. Non-limiting examples include, PDGF (Platelet Derived Growth Factor), rhPDGF-BB (Becaplermin), EGF (Epidermal Growth Factor), PDECGF (Platelet Derived Endothelial Cell Growth Factor), aFGF (Acidic Fibroplast Growth Factor), bFGF (Basic Fibroplast Growth Factor), TGF-alpha (Transforming Growth Faxtor alpha), TGF-beta (Transforming Growth Factor beta), KGF (Keratinocyte Growth Factor), VEGF (Vascular endothelial growth factor), IGF1/IGF2 (Insulin-Like Growth Factor), collagen, hyaluronic acid, protease inhibitors such as; alpha-1 antitrypsin and SLPI (Secretory Leukocyte Protease Inhibitor), and antibacterial proteins or peptides directed specifically against undesirable, putrefactive bacteria.

A “wound healing-promoting substance” is in preferred embodiments a bioactive agent produced by a lactic acid bacteria. The wound healing-promoting substance can either be a recombinantly expressed protein or an agent naturally expressed by the lactic acid bacteria. Examples of such naturally lactic acid bacteria bioactive agents include, but is not limited to, any bioactive agent capable of inhibiting the growth of putrefactive, putrefactive microorganisms through other metabolic products such as e.g. hydrogen peroxide.

Preferred metabolites produced by lactic acid producing bacteria according to the present invention that are believed to exert antagonistic action(s) against putrefactive microorganisms, as well as their mode of action, are summarized in the below Table 1.

TABLE 1
Antagonistic activities caused by preferred
lactic acid producing bacteria

| Bioactive agent | [symbol] promotion of wound healing |
|---|---|
| 1. Diacetyl | Antimicrobial agent |
| 2. Hydrogen peroxide/ | Antibacterial by oxidation |
| Lactoperoxidase | |
| 3. Lactic acid | Lactic acid lowers the pH which results |
| | in reduced activity of many proteases in |
| | the wound and consequently a |
| | restoration of the balance between |
| | anabolic and catabolic processes in the |
| | wound. |
| 4. Bacteriocins | Inhibit the formation of bacterial cell |
| | wall or membrane, e.g. by poreforming |
| | or by inhibition of the synthesis of |
| | membrane/cell wall components. |

[symbol]

indicates data missing or illegible when filed

Yet another example of a lactic acid bacteria bioactive agents believed to be capable of promoting wound healing is B-vitamins. Experiments on fermented milk products have revealed that lactic cultures require B-vitamins for their metabolic activities. However, some lactic cultures synthesize B-vitamin 16 which—according to one presently preferred hypothesis—is believed to be involved in the promotion of wound healing.

It should be noted that B-vitamin contents of fermented milk products are believed to be a function of both species as well as the strain of lactic acid bacteria used in the manufacture of the vitamin in question. Similarly, vitamins are synthesized by the lactic cultures in the gut microflora, in symbiosis with other flora.

“Treatment” as used herein refer equally to curative therapy, prophylactic therapy, and preventative therapy. The term includes an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent, or with the bioactive agent in combination with a pharmaceutical composition of the present invention.

“Pharmacologically effective amount”, “pharmaceutically effective amount” or “physiologically effective amount of a “bioactive agent” is the amount of an active agent present in a pharmaceutical composition as described herein that is needed to provide a desired level of active agent at the site of action in an individual to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of a bioactive agent can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

The terms “enhancing” and “improving” a beneficial effect, and variations thereof, as used herein, refers to the therapeutic effect of the bioactive agent against placebo, or an increase in the therapeutic effect of a state-of-the-art medical treatment above that normally obtained when a pharmaceutical composition is administered without the bioactive agent of this invention. “An increase in the therapeutic effects” is manifested when there is an acceleration and/or increase in intensity and/or extent of the therapeutic effects obtained as a result of administering the bioactive agent(s). It also includes extension of the longevity of therapeutic benefits. It can also manifest where a lower amount of the pharmaceutical composition is required to obtain the same benefits and/or effects when it is co-administered with bioactive agent(s) provided by the present invention as compared to the administration in a higher amount of the pharmaceutical composition in the absence of bioactive agent. The enhancing effect preferably, but not necessarily, results in treatment of acute symptoms for which the pharmaceutical composition alone is not effective or is less effective therapeutically. Enhancement is achieved when there is at least a 5% increase in the therapeutic effects, such as at least 10% increase in the therapeutic effects when a bioactive agent of the present invention is co-administered with a pharmaceutical composition compared with administration of the pharmaceutical composition alone. Preferably the increase is at least 25%, more preferably at least 50%, even more preferably at least 75%, most preferably at least 100%.

“Co-administering” or “co-administration” of bioactive agent(s), or bioactive agents and state-of-the-art medicaments, as used herein, refers to the administration of one or more bioactive agents of the present invention, or administration of one or more bioactive agents of the present invention and a state-of-the-art pharmaceutical composition within a certain time period. The time period is preferably less than 24 hours, such as less than 12 hours, for example less than 6 hours. such as less than 3 hours. However, these terms also mean that the bioactive agent and a therapeutic composition can be administered together.

“Individual” refers to vertebrates, particular members of the mammalian species, and includes, but is not limited to domestic animals, such as cattle, horses, pigs, sheep, mink, dogs, cats, mice, guinea pigs, rabbits, rats; sports animals, such as horses, poly ponies, dogs, camels, and primates, including humans.

### BRIEF DESCRIPTION OF THE DRAWING

Schematic drawings of different, non-limiting embodiment of the present invention are illustrated in FIG. 1.

FIG. 1, panel A: A wound dressing that allows the metabolites of the lactic bacteria to be delivered to the wound. Lyophilized lactic acid producing bacteria are contained in a membrane bag that allows for the passage of proteins but not for the bacteria. On top of the bacteria containing bag is attached an absorbing pad and a topfilm to avoid leakage of moisture. An adhesive surface is applied on the edges of the topfilm.

FIG. 1, panel B: A wound dressing that allows for the lactic acid bacteria to be released into the wound. Capsules containing lyophilized lactic acid producing bacteria are distributed into an absorbing pad. As the pad absorbs fluid, the capsules will disintegrate and release the bacteria into the wound.

FIG. 1, panel C: In a dual chamber syringe, one chamber is pre-filled with a hydrogel and the other chamber is pre-filled with lyophilized lactic acid producing bacteria. When the plunger is pressed the membrane closing the chambers are broken and the hydrogel and lactic acid bacteria from the two chambers are mixed in the mixing chamber before exiting the syringe.

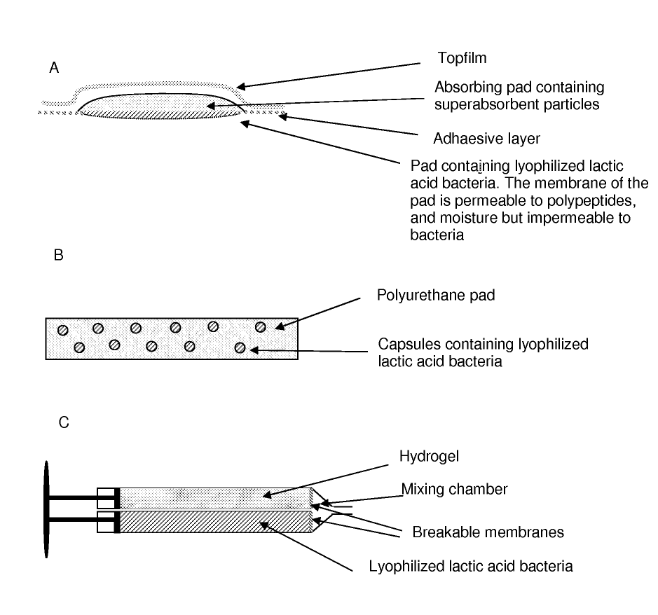

### DETAILED DESCRIPTION OF THE INVENTION

The present invention in one embodiment is directed to a wound or tissue dressing comprising lactic acid bacteria. The wound or tissue dressing can further comprise an absorbent compound for absorbing wound exudate, wherein the lactic acid bacteria are either attached to or comprised in the absorbent compound.

### Absorbent Compound

The absorbent compound can be any compound capable of absorbing wound exudate.

In one embodiment the absorbent compound comprises or consists of a hydrogel forming material. The hydrogel forming material can form an amorphous hydrogel, but the hydrogel forming material can also be in the form of e.g. a sheet—in which case the dressing will be a hydrogel sheet dressing.

In other embodiments, the absorbent compound of the wound or tissue dressing comprises or consists of a hydrocolloid forming material.

The absorbent compound can comprise or consist of a porous polymer suitable for entry of wound exudate therein, i.e. the capillary force allows wound exudate to enter into the porous polymer. The porous polymer is often hydrophilic or sufficiently hydrophilic to allow transport of wound extrudate.

In a still further embodiment the absorbent compound comprises or consists of a foam forming material.

It is important that the absorbent compound is in fluid contact with the wound e.g. through a gel or a matrix, such as a scaffold, or, alternatively, that the absorbent compound can contact the wound directly.

The porous material of the absorbent compound can be bioabsorbable and be adapted for serving as scaffold for new cells to migrate into and proliferate. Such a “connective” absorbent scaffold can remain in place on the wound bed throughout the healing process, and later be absorbed and replaced by new tissue. During the wound healing process, the connective absorbent compound will transmit wound exudate from the wound bed to the bioabsorbable and/or porous material of the absorbent compound.

The lactic acid bacteria can be attached, covalently or non-covalently, to the gel or matrix of the bioabsorbable and/or porous material of the absorbent compound, or the lactic acid bacteria can be present in, i.e. mixed with, the bioabsorbable and/or porous material of the absorbent compound. In one embodiment the lactic acid bacteria are contained in a compartment separated from the bioabsorbable and/or porous material of the absorbent compound.

In one embodiment, when being prepared for use, a breakable barrier sealing the wound or tissue dressing from an external environment is broken and the lactic acid bacteria are brought into contact with the bioabsorbable and/or porous material of the absorbant compound—and over time the lactic acid bacteria are also brought into contact with wound exudate and the wound environment itself.

The absorbent compound can be a material that is absorbent to liquid while at the same time serves as a barrier for cell penetration. Such an absorbent compound can be referred to as an “absorbent barrier material”. An absorbent barrier material can e.g. prevent lactic acid bacteria present in the bioabsorbable and/or porous material of the absorbent compound from entering the wound itself. However, bioactive agents produced by the lactic acid bacteria and having wound healing promoting abilities are allowed to enter the wound area.

Besides absorbing wound exudate and preventing undesirable bacteria from entering the wound, the absorbent compound can also act as a reservoir for liquids to hydrate the wound. The features of non-adhesion and resistance to penetration by cells provide the important advantage that the absorbent barrier material—and any subsequent connective compound—is easily removed and/or replaced as needed without causing trauma to growing cells or tissue.

If desirable, the absorbent compound can be in contact with a further compound, such as a breathable film that can serve as a barrier to the entry of contaminants into the wound bed. One example of such a barrier is a topfilm.

The absorbent compound can be any material approved for wound care. Materials that can be used as an absorbent compound include fabrics, foams or fibres of e.g. polyester, polyurethans, polypropylenes and polyethylenes which are optionally bonded to polyester film (such as Kendall's Novenette). Other suitable materials include, but are not limited to, natural and synthetic polymeric absorbents, hydrocolloids, superabsorbents, and cellulosic absorbents. Cellulosic materials include cotton, rayon, wood and cellulose.

The absorbent compound can be a superabsorbent material in any suitable form. Typical superabsorbents include starch grafted copolymers of acrylate salts, starch grafted copolymers of acrylamide salts, polyacrylate salts and the like, including mixtures thereof.

Superabsorbent compounds and composites are easily prepared or commercially available. Once such product is the composite air laid superabsorbent pad (dry forming process and the superabsorbent fiber flock SAFF) sold by Hanfspinnern Steen & Company. The superabsorbent may also be a delayed released web superabsorbent.

Superabsorbent webs that may be used in the present invention to serve as, or to be incorporated into, the absorbent compound can also include carded or random webs made from, for example, cotton, rayon, polyethylene, polyester, or wool. Another suitable web is a spun-laced web made from polyester, polypropylene, or polyethylene. The superabsorbent webs may also be in the form of tissues either single ply or multiple ply and either creped or uncreped. Delnet, a product of Applied Extrusion Technologies which consists of a range of materials manufactured from polyethylene or polypropylene using extrusion embossing and orientation processes may also be used as a web for preparing a superabsorbent web.

Superabsorbent webs can be formed by any convenient means, e.g., by slightly moistening or misting a web. After misting, a powdered superabsorbent may be applied followed by running the web through a dry oven or heating the roll. The powder adjacent to the moistened web will become tacky and adhere to the adjacent material (fiber, surface), and the loose powder would then be vacuumed off.

Alternatively, superabsorbent powder can be sandwiched between non-woven webs/paper and subjected to moist steam which would make the superabsorbent tacky so that it would then stick to adjacent surfaces. The sandwiched superabsorbent and web would then be dried, creating a two-ply web with superabsorbent between them. The superabsorbent connective compound can also be heat bonded to the other connective compounds.

The wound or tissue dressing according to the present invention can contain from about 5% to about 50% by weight of water, such as from about 5% to about 40% by weight of water, for example from about 5% to about 30% by weight of water, such as from about 5% to about 25% by weight of water, for example from about 5% to about 20% by weight of water, such as from about 5% to about 15% by weight of water, for example from about 5% to about 10% by weight of water, such as from about 10% to about 40% by weight of water, for example from about 10% to about 30% by weight of water, such as from about 10% to about 25% by weight of water, for example from about 10% to about 20% by weight of water, such as from about 10% to about 15% by weight of water, such as from about 15% to about 40% by weight of water, for example from about 15% to about 30% by weight of water, such as from about 15% to about 25% by weight of water, for example from about 15% to about 20% by weight of water.

### Adhesive Surface

The absorbent compound can comprise at least one adhesive surface suitable for contacting a wound or the absorbent compound can be attached to at least one adhesive surface suitable for contacting a wound. When the absorbent compound is attached to at least one adhesive surface suitable for contacting a wound the absorbent compound and the adhesive surface are most often manufactured separately and only brought together during the manufacturing of the wound or tissue dressing according to the present invention. The adhesive surface can simply be positioned on or spread out over the corresponding surface of the absorbent compound, such as the absorbent compound surface which is going to be aligned with the surface of a wound.

The at least one adhesive surface can be separated from the absorbent compound by a permeable or semi-permeable barrier allowing wound extrudate to be diverted from the wound to the absorbent compound. Alternatively, the at least one adhesive surface can itself comprise a barrier acting as a permeable or semi-permeable barrier that allows wound extrudate to be diverted from the wound to the absorbent compound.

The absorbent compound can also be attached to a topfilm at least partly sealing the absorbent compound from the external environment. Alternatively, the absorbent compound itself comprises a functionality acting as a topfilm at least partly sealing the absorbent compound from the external environment.

The topfilm is often porous and the topfilm can comprise an oxygen- and vapor-permeable layer permitting transpiration of liquid from the absorbent compound.

### Gelatin and Collagen Absorbent Compounds

In some embodiments the wound or tissue dressing according to the present invention comprises an absorbent compound comprising or consisting of gelatin and/or collagen, including a combination of gelatin and collagen.

When the absorbent compound comprises or consists of gelatin, the gelatin can be cross-linked and form a matrix, such as a matrix in the form of a hydrogel.

Alternatively, the wound or tissue dressing can comprise or consist of gelatin which is not crosslinked. The gelatin can be in granulated or particulated form and most often such dressings employ hydrocolloids.

When the absorbent compound comprises or consists of collagen the collagen can be cross-linked and form a matrix, such as a matrix in the form of a hydrogel.

Alternatively, the wound or tissue dressing can comprise or consist of collagen which is not crosslinked. The collagen can be in granulated or particulated form and most often such dressings employ hydrocolloids.

Hyaluronic acid can be present in the dressing in combination with any or both of gelatin and collagen. In one embodiment, there is provided an absorbant compound comprising a biologically absorbable material and hyaluronic acid, or a derivative thereof.

Hyaluronic acid is a natural heteropolysaccharide consisting of alternate residues of D-glucuronic acid and N-acetyl-D-glucosamine. It is a linear polymer having a molecular weight ranging from about 50 to about 13,000 kDa, depending on the source it is obtained from and on the method of preparation. Hyaluronic acid is the main component of the extracellular matrix, but it has other functions such as hydration of tissues, lubrication as well as cell migration and differentiation. A suitable molecular weight for hyaluronic acid for the purposes described herein will be in the range of from 50 to 5,000 kDa, such as in the range of from 50 to 4,000 kDa, e.g. in the range of from 100 to 3,000 kDa. In a particular preferred embodiment of the invention, the hyaluronic acid, or a derivative thereof, has a molecular weight in the range of from 250 to 3,500 kDa, more preferably in the range of from 500 to 2,500 kDa, such as in the range of from 500 to 2,000 kDa.

Optionally, the hyaluronic acid molecule may be cross-linked, e.g. by chemical or physical means. In a preferred embodiment of the invention the employed hyaluronic acid is pH neutral, i.e. an aqueous solution of the employed HA exhibits a pH value in the range of from 5 to 9, preferably in the range of from 6-8, in particular in the range of from 6.5 to 7.5, such as about 7. The hyaluronic acid used in the present invention may be extracted from any source, for example from rooster comb. Alternatively hyaluronic acid may be obtained by fermentation.

Derivatives of hyaluronic acid include, for example, esters of hyaluronic acid, as well as the derivatives described in U.S. Pat. No. 5,356,883; U.S. Pat. No. 6,548,081; U.S. Pat. No. 4,851,521; U.S. Pat. No. 6,027,741; US 2003 181689; EP 1 095 064; EP 0 341 745; WO 02/18450 and WO 2004/035629. In addition, the term “derivative” is also intended to cover hyaluronate salt, including, but not limited to, sodium hyaluronate, potassium hyaluronate, magnesium hyaluronate and calcium hyaluronate.

### Alginate Absorbent Compounds

In one embodiment the absorbent compound comprises an optionally cross-linked alginate compound, such as an alginate ester, for example an alginate ester comprising propylene glycol alginate.

The degree of esterification of the alginate ester is typically from 35% to 95% and the absorbent compound can contain from 10% to 25% by weight of the alginate ester. Reference is made to U.S. Pat. No. 6,022,556 and U.S. Pat. No. 5,735,812, both of which are incorporated herein by reference.

### Wound Healing-Promoting Substance(s)

The invention in some embodiments is directed to dressings and methods further comprising one or more wound healing-promoting substance(s) either added as proteins or supplied by recombinantly expression by the lactic acid bacteria. Such substances are e.g. capable of promoting the healing of slow-healing or chronic wounds. Examples include alpha-1 antitrypsin, SLPI (Secretory Leukocyte Protease Inhibitor), PDGF (Platelet Derived Growth Factor), rhPDGF-BB (Becaplermin), EGF (Epidermal Growth Factor), PDECGF (Platelet Derived Endothelial Cell Growth Factor), aFGF (Acidic Fibroplast Growth Factor), bFGF (Basic Fibroplast Growth Factor), TGF-alpha (Transforming Growth Faxtor alpha), TGF-beta (Transforming Growth Factor beta), KGF (Keratinocyte Growth Factor), IGF1/IGF2 (Insulin-Like Growth Factor), VEGF (Vascular endothelial growth factor).

### Lactic Acid Bacteria

Lactic acid bacteria produce lactic acid from lactose by means of enzymes such as beta-galactosidases, glycolases and lactic dehydrogenases (LDH).

A lactic acid bacteria in accordance with the present invention can be any bacteria capable of producing lactic acid—and actually producing lactic acid and/or another desirable bioactive agent, such as a protease, under practical circumstances when employed in connection with the claimed wound or tissue dressing.

Lactic acid producing bacteria according to the present invention are preferably selected from the group consisting of Carnobacterium, Enterococcus, Lactobacillus, Lactococcus, Leuconostoc, Oenococcus, Pediococcus, Streptococcus, Tetragenococcus, Vagococcus and Weissella. In one embodiment, Bacillus coagulans is excluded from the above definition of lactic acid producing bacteria.

The skilled person will be aware that the genus Lactobacillus remains heterogeneous with over 60 species (ymol % G+C content ranging from 33 to 55). Lactobacilli are gram positive and vary in morphology from long, slender rods to short coccobacilli, which frequently form chains. Their metabolism is fermentative; some species are aerotolerant and may utilize oxygen through the enzyme flavoprotein oxidase, while others are strictly anaerobic. While spore bearing lactobacilli are facultative anaerobes, the rest are strictly anaerobic. The growth is optimum at pH 5.5 to 5.8 and the organisms have complex nutritional requirements for amino acids, peptides, nucleotide bases, vitamins, minerals, fatty acids and carbohydrates.

The genus Lactobacillus is generally divided into three groups based on fermentation patterns:

1. homofermentative: produce more than 85% lactic acid from glucose.

2. heterofermentative: produce only 50% lactic acid and considerable amounts of ethanol, acetic acid and carbon dioxide.

3. less well known heterofermentative species which produce DL-lactic acid, acetic acid and carbon dioxide.

Particularly preferred species of Lactobacillus include L. sporogenes; L. acidophilus; L. plantarum; L. casei; L. brevis; L. delbruckii and L. lactis.

According to one presently preferred hypothesis, the lactic acid forming bacteria according to the present invention are capable of counter-acting a delayed wound healing process caused by bacterial infection of a wound, as described in more detail herein below.

Apart from causing pain, swelling and odour problems for the patient, putrefactive bacteria also affect the wound healing process through the production of proteases and toxins, as well as by promoting a chronic inflammatory state. In the inflammatory state, cells of the tissue (e.g. neutrophils) release numerous proteases. Proteases from said putrefactive bacteria as well as from inflammatory cells can be expected to digest growth factors and other desirable proteins that would otherwise have been able to promote the wound healing process.

The activity of a protease is likely to be dependent on the pH of the environment in which the protease is to exert its activity (Schultz et al, 2005). Many proteases (e.g. elastase and plasmin) have a pH optimum at about pH 8. Open wounds tend to have a neutral or alkaline pH, predominantly in the range of from 6.5 to 8.5 (Dissemond et al, 2003). Chronic wounds are known to have permanently elevated protease levels and one strategy in accordance with the present invention for promoting the wound healing process is to modulate and preferably reduce the proteolytic activity of undesirable proteases by using a pH modulator, such as the lactic acid produced by the lactic acid forming bacteria present in the wound or tissue dressing according to the present invention.

Accordingly, one beneficial action of the lactic acid bacteria according to the present invention is their ability to modulate the activity of undesirable polypeptides present in a wound which has been infected by putrefactive bacteria.

The levels of optical isomeric forms of lactic acid produced by lactic acid forming bacteria depend upon the nature of the culture. The structural configurations of these isomers are as follows:

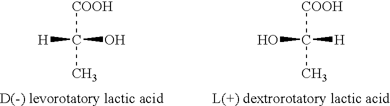

Both forms may be relevant in connection with the present invention. In humans, both isomers are absorbed from the intestinal tract. Whereas L(+) lactic acid is completely and rapidly metabolized in glycogen synthesis, D(−) lactic acid is metabolized at a lesser rate, and the unmetabolized acid is excreted in the urine. As an example, L. acidophilus produces the D(−)-form, whereas L. sporogenes produces only L(+)-lactic acid.

It is furthermore believed that some of the more volatile acids produced during lactic acid bacteria fermentation may also possess some antimicrobial activity, e.g. under conditions of low oxidation-reduction potential.

Lactococcus as used herein refer collectively to a lactic acid bacterial genus of five major species. They are typically spherical or ovoid, 0.5 to 1.2 μm by 0.5 to 1.5 μm, and occur in pairs and short chains. They are non-spore forming and are not motile. The type species for the genus is L. lactis. Lactococcus differ from other lactic acid bacteria as they have pH, salt and temperature tolerances for growth.

The new genus Weissella has been established to include one member of the genus Leuconostoc (Leuc. paramesenteroides) and heterofermentative lactobacilli with unusual interpeptide bridges in the peptidoglycan. Contrary to the clear-cut division of the streptococci, morphological and physiological features of Weissella do not directly support this grouping which now incorporates species that produce D(−)-lactate as well as DL-lactate.

The new genus Carnobacterium is morphologically similar to the lactobacilli, but it shares some physiological similarities (e.g. growth at pH 9.5) and a common phylogenetic branch with the genus Enterococcus.

The wound or tissue dressing according to the present invention can contain—in one embodiment—from 10⁰ to 10⁹ lactic acid producing bacteria per cm³, such as from 10¹ to 10⁶ lactic acid producing bacteria per cm³, for example from 10² to 10⁴ lactic acid producing bacteria per cm³.

### Biodegradable Microspheres

The optionally lyophilized lactic acid producing bacteria can be encapsulated or formulated as a gel—e.g. in gelatin or collagen—with a view to stabilizing the lactic acid producing bacteria. In one embodiment, the optionally lyophilized lactic acid producing bacteria are formulated as a lipid and/or polymer-comprising microsphere which includes at least one biodegradable polymer.

The biodegradable polymers can be homopolymers, such as polylactides, polyglycolides, poly(p-dioxanones), polycaprolactones, polyhydroxyalkanoates, polypropylenefumarates, polyorthoesters, polyphosphate esters, polyanhydrides, polyphosphazenes, polyalkylcyanoacrylates, polypeptides, or genetically engineered polymers. At the same time, the biodegradable polymers can be copolymers (random or block) such as poly(lactide-glycolides), poly(p-dioxanone-lactides), poly(p-dioxanone-glycolides), poly(p-dioxanone lactide-glycolides), poly(p-dioxanone-caprolactones), poly(p-dioxanone-alkylene carbonates), poly(p-dioxanone-alkylene oxides), poly(p-dioxanone-carbonate-glycolides), poly(p-dioxanone-carbonates), poly(caprolactone-lactides), poly(caprolactone-glycolides), poly(hydroxyalkanoates), poly(propylenefumarates), poly(orthoesters), poly(ether-esters), poly(ester-amides), poly(ester-urethanes), polyphosphate esters, polyanhydrides, poly(ester-anhydrides), polyphosphazenes, polypeptides and genetically engineered copolymers. The lipids of the microspheres, when present, can be zwitterionic lipids, acidic lipids, cationic lipids, sterols, or triglycerides of many types, including many phospholipids.

A biodegradable polymer according to the present invention is one that can be degraded to a low molecular weight and may or may not be eliminated from a living organism. The products of biodegradation may be the individual monomer units, groups of monomer units, molecular entities smaller than individual monomer units, or combinations of such products. Such polymers may also be metabolized by organisms. Biodegradable polymers can be made up of biodegradable monomer units. A biodegradable compound is one that can be acted upon biochemically by living cells or organisms, or parts of these systems, or reagents commonly found in such cells, organisms, or systems, including wound exudates and similar aqueous compositions, including water, and broken down into lower molecular weight products. An organism can play an active or passive role in such processes.

The biodegradable polymer chains useful in the present invention preferably have molecular weights in the range of from 500 to 5,000,000. The biodegradable polymers can be homopolymers, or random or block copolymers. The copolymer can be a random copolymer containing a random number of subunits of a first copolymer interspersed by a random number of subunits of a second copolymer. The copolymer can also be block copolymer containing one or more blocks of a first copolymer interspersed by blocks of a second copolymer. The block copolymer can also include a block of a first copolymer connected to a block of a second copolymer, without significant interdispersion of the first and second copolymers.

Biodegradable homopolymers useful in the invention can be made up of monomer units selected from the following groups: hydroxy carboxylic acids, such as alpha-hydroxy carboxylic acids, including lactic acid, glycolic acid, lactide (intermolecularly esterified dilactic acid), and glycolide (intermolecularly esterified diglycolic acid); beta-hydroxy carboxylic acids, including beta-methyl-beta-propiolactone; gamma-hydroxy carboxylic acids, delta-hydroxy carboxylic acids; and epsilon-hydroxy carboxylic acids, including epsilon-hydroxy caproic acid; lactones, such as: beta-lactones; gamma-lactones; delta-lactones, including valerolactone; and epsilon-lactones, such as epsilon-caprolactone; benzyl ester-protected lactones, such as benzyl malolactone; lactams, such as: beta-lactams; gamma-lactams; delta-lactams; and epsilon-lactams; thiolactones such as 1,4-dithiane-2,5-dione; dioxanones; unfunctionalized cyclic carbonates such as: trimethylene carbonate, alkyl substituted trimethylene carbonates, and spiro-bis-dimethylene carbonate (2,4,7,9-tetraoxa-spiro[5.5]undecan-3,8-dione); anhydrides; substituted N-carboxy anhydrides; propylene fumarates; orthoesters; phosphate esters; phosphazenes; alkylcyanoacrylates; aminoacids; polyhydroxybutyrates; and substituted variations of the above monomers.

### Hydrocolloids

The wound or tissue dressing can comprise a hydrocolloid, but in some embodiments the hydrocolloid can be omitted. In embodiments wherein a hydrocolloid is used, the hydrocolloid comprises about 20 to about 60 weight percent of the wound or tissue dressing, based on total weight.

The hydrocolloid can comprise e.g. from about 25 to about 55 weight percent of the composition, such as from about 30 to about 50 weight percent of the composition. In one embodiment, the hydrocolloid comprises about 40 weight percent of the composition.

The hydrocolloid used in the present invention can be synthetically prepared or naturally occurring. Varieties of hydrocolloids within the scope of the present invention include synthetic polymers prepared from single or multiple monomers, naturally occurring hydrophilic polymers, or chemically modified naturally occurring hydrophilic polymers. It is preferred that the hydrocolloid is dermatologically acceptable and non-reactive with the skin of the patient or other components of the composition. Preferred examples are hydrocolloids comprising gelatin and/or collagen.

Further specific examples include hydrocolloids comprising e.g. polyhydroxyalkyl acrylates and methacrylates, polyvinyl lactams, polyvinyl alcohols, polyoxyalkylenes, polyacrylamides, polyacrylic acid, polystyrene sulfonates, natural or synthetically modified polysaccharides, alginates, gums, and cellulosics and modified celluloses.

Representative polysaccharides include e.g. starch, glycogen, hemicelluloses, pentosans, celluloses, pectin, chitosan, and chitin.

Representative gums include e.g. Arabic, Locust Bean, Guar, Agar, Carrageenan, Xanthan, Karaya, tragacanth, Ghatti, and Furcelleran gums.

Representative modified celluloses include methyl cellulose, hydroxypropyl methyl cellulose, carboxymethylcellulose, and hydroxypropyl cellulose.

Hydrocolloids which are water soluble or swellable hydrocolloids can be selected e.g. from the group consisting of polyvinyl alcohols, powdered pectin, methyl cellulose, hydroxypropyl methyl cellulose, carboxymethylcellulose, hydroxypropyl cellulose and mixtures thereof.

Further examples of suitable hydrocolloids include synthetic polymers that may be either linear or crosslinked. Non-limiting examples of synthetic hydrocolloids include e.g. polymers prepared from N-vinyl lactams, e.g. N-vinyl-2-pyrrolidone, 5-methyl-N-vinyl-2-pyrrolidone, 5-ethyl-N-vinyl-2-pyrrolidone, 3,3-dimethyl-N-vinyl-2-pyrrolidone, 3-methyl-N-vinyl-2-pyrrolidone, 3-ethyl-N-vinyl-2-pyrrolidone, 4-methyl-N-vinyl-2-pyrrolidone, 4-ethyl-N-vinyl-2-pyrrolidone, N-vinyl-2-valerolactam, and N-vinyl-2-caprolactam.

Other monomers useful to prepare a synthetic hydrocolloid include hydroxyalkyl acrylates and methacrylates, (such as 2-hydroxyethyl acrylate, 2-hydroxyethyl methacrylate, 2-hydroxypropyl acrylate, 2-hydroxypropyl methacrylate, 2,3-dihydroxypropyl methacrylate), acrylic acid, methacrylic acid and a tertiary amino-methacrylimide, (e.g. trimethylamino-methacrylimide), crotonic acid, and pyridine. Additional monomers useful to prepare a synthetic hydrocolloid include water soluble amides, (such as N-(hydroxymethyl)acrylamide and -methacrylamide, N-(3-hydroxypropyl)acrylamide, N-(2-hydroxyethyl)methacrylamide, N-(1,1-dimethyl-3-oxabutyl)acrylamide N-[2-(dimethylamine)ethyl]acrylamide and -methacrylamide, N-[3-(dimethylamino)-2-hydroxylpropyl]methacrylamide, and N-[1,1-dimethyl-2-(hydroxymethyl)-3-oxabutyl]acrylamide); water-soluble hydrazine derivatives, (such as trialkylamine methacrylimide, and dimethyl-(2-hydroxypropyl)amine methacrylimide); mono-olefinic sulfonic acids and their salts, (such as sodium ethylene sulfonate, sodium styrene sulfonate and 2-acrylamideo-2-methylpropanesulfonic acid); and the following monomers containing nitrogen in the non-cyclic or cyclic backbone of the monomer: 1-vinyl-imidazole, 1-vinyl-indole, 2-vinyl imidazole, 4(5)-vinyl-imidazole, 2-vinyl-1-methyl-imidazole, 5-vinyl-pyrazoline, 3-methyl-5-isopropenyl-pyrazole, 5-methylene-hydantoin, 3-vinyl-2-oxazolidone, 3-methacrylyl-2-oxazolidone, 3-methacryl)-1-5-methyl-2-oxazolidone, 3-vinyl-5-methyl-2-oxazolidone, 2- and 4-vinyl-pyridine, 5-vinyl-2-methyl-pyridine, 2-vinyl-pyridine-1-oxide, 3-isopropenyl-pyridine, 2- and 4-vinyl-piperidine, 2- and 4-vinyl-quinoline, 2,4-dimethyl-6-vinyl-s-triazine, and 4-acrylyl-morpholine.

### Hydrogels

Cross-linking of the linear polymer chains of the hydrocolloid may be desired to improve cohesive properties of the gel dispersed in the pressure sensitive adhesive matrix. When such crosslinking is desired for polymers made from vinyl monomers discussed above, a multi-ethylenically unsaturated compound with the ethylenic groups being vinyl, allyl, or methallyl groups bonded to nitrogen, oxygen or carbon atoms can be used.

Non-limiting examples of cross-linking agents for vinyl containing polymers include divinyl, diallyl, or dimethallyl esters (e.g. ethylene glycol dimethacrylate, divinyl succinate, divinyl adipate, divinyl maleate, divinyl oxalate, divinyl malonate, divinyl glutarate, diallyl itaconate, diallyl maleate, diallyl fumarate, diallyl diglycolate, diallyl oxalate, diallyl adipate, diallyl succinate, diallyl azelate, diallyl malonate, diallyl glutarate, dimethallyl maleate, dimethallyl oxalate, dimethallyl malonate, dimethallyl succinate, dimethallyl glutarate, and dimethallyl adipate); divinyl, diallyl or dimethallyl ethers (e.g. diethyleneglycol divinyl ether, butane diol divinyl ether, ethylene glycol divinyl ether, ethylene glycol diallyl ether, diethylene glycol diallyl ether, butane diol diallyl ether, ethylene glycol dimethallyl ether, diethylene glycol dimethallyl ether, and butane diol dimethallyl ether); divinyl, diallyl or dimethallyl amides including bis(N-vinyl lactams), (e.g., 3,3′-ethylene bis(N-vinyl-2-pyrrolidone) and methylene-bis-acrylamide); and divinyl, diallyl and dimethallyl ureas.

Preferred cross-linking agents include ethylene glycol dimethacrylate, methylene-bis-acrylamide, diallyl maleate, and 3,3′-ethylidene bis(N-vinyl-2-pyrrolidone). For n-vinyl lactams, the preferred crosslinking agents are diallyl maleate and 3,3′-ethylidene bis (N-vinyl-2-pyrrolidone). For acrylates and methacrylates, the preferred crosslinking agents are ethylene glycol dimethacrylate and methylene-bis-acrylamide.

### Humectants

The dressing can also contain a humectant to reduce the partial vapor pressure of the water in the wound or tissue dressing or to reduce the rate at which the wound or tissue dressing dries out. Suitable humectants are miscible with water to a large extent and are generally suitable for application to the skin.

The humectant can be e.g. glycerol and propylene glycol and the absorbent compound typically contains from about 10% to about 90% by weight of the humectant.

Polyols are especially suitable for the purpose and suitable polyols may include monopropylene glycol or glycerin (glycerol). The polyol may be present in proportions of 20 to 50% (by weight) of the total formulation; alternatively the range is 30 to 40%. This relatively high proportion of polyol also ensures that if the paste should dry out to any degree, the resulting paste remains soft and flexible because the glycerin may act as a plasticiser for the polymer. When the paste is applied on a bandage, for example, it may therefore still be removed easily from the skin when the paste has lost water without the need to cut the bandage off. The polyol also has the advantage of functioning to prevent the proliferation of bacteria in the paste when it is in contact with the skin or wound, particularly infected wounds.

### Further Bioactive Compounds

The formulation can include other ingredients such as antibacterial agents, antifungal agents, anti-inflammatory agents, and the like. Other ingredients may also be found suitable for incorporation into the formulation.

### Outer Packaging Comprising a Sterile Barrier Seal

The “sterile” dressings according to the present invention can be protected from undesirable contamination by an outer packaging comprising a sterile barrier seal in the form of an unbroken seal separating the “sterile” dressing from an external, non-sterile environment. The sterile barrier seal is only broken immediately prior to use in order to retain the “sterility” of the dressing according to the present invention.

The outer packaging may be pelable or otherwise removable from the outer surface of the dressing. The dressing is preferably enclosed in an outer packaging of a flexible, semi-rigid, or rigid plastic and/or metallic film providing a sterile barrier. The outer packaging typically consists of materials selected from the group consisting of plastics, aluminium foils and plastic laminates, where the plastic is preferably selected from the group consisting of PET, PE, LLDPE, CPP, PA, PETP, METPET, Tyvek, said plastic being optionally bonded with an adhesive (e.g. Polyurethane) or co-extruded. The outer packaging preferably forms a complete barrier to moisture.

A particularly interesting embodiment of the outer packaging includes a pouch of laminated foil. The laminate may be PET, such as PET having a thickness of approximately 12 microns.

### Method for Manufacturing Wound or Tissue Dressings According to the Invention

The present method is also directed to a method for manufacturing the wound or tissue dressing according to the invention, said method comprising the steps of providing an optionally encapsulated and/or lyophilized lactic acid producing bacteria, mixing or attaching said lactic acid producing bacteria with the wound or tissue dressing, or with the absorbent compound of the wound or tissue dressing, thereby obtaining a wound or tissue dressing according to the invention.

The method can comprise the further step of providing the absorbent compound with at least one adhesive surface suitable for contacting a wound, or the further step of attaching at least one adhesive surface suitable for contacting a wound to the absorbent compound.

In another further step there is provided a permeable or semi-permeable barrier for separating the at least one adhesive surface from the absorbent compound by introducing said permeable or semi-permeable barrier between the absorbent compound and the at least one adhesive surface, wherein said permeable or semi-permeable barrier allows wound exudate to be diverted from the wound to the absorbent compound.

In a yet further step the method comprises providing a permeable or semi-permeable barrier capable of partly separating—during use—the at least one adhesive surface from the wound by introducing said permeable or semi-permeable barrier on the surface of the adhesive surface, wherein said permeable or semi-permeable barrier—during use—allows wound exudate to be diverted from the wound to the absorbent compound through the adhesive surface.

In yet another step, the permeable or semi-permeable barrier will allow proteins and exudate to pass, but will retain the lactic acid producing bacteria in the dressing.

In yet another further step a topfilm can be provided and attached to the absorbent compound, wherein said topfilm seals at least partly the absorbent compound from the external environment. The absorbent compound can also comprise a topfilm as an integrated part, wherein said topfilm at least partly seals the absorbent compound from the external environment. The topfilm can be porous or non-porous. In one embodiment, the topfilm comprises an oxygen- and vapor-permeable layer permitting transpiration of liquid from the absorbent compound.

In order to kept the moisture of the dressing low, the dressing can be packed with a desiccant, such as silica.

### Wound Treatment Methods

Various uses of the wound or tissue dressings according to the present invention are envisaged. In one embodiment there is provided a method for treating a wound in an individual, said method comprising the steps of contacting said wound with the wound or tissue dressing according to the present invention, and treating the wound.

The treatment can in principle result in healing of the wound or in accelerated healing of the wound. The accelerated healing can be a result of e.g. administration of a wound-healing promoting substance, or a bioactive agent produced by the lactic acid bacteria. The wound-healing substance and/or the bioactive agent can be produced naturally or recombinantly by the lactic acid bacteria. Alternatively, the wound healing can be promoted by preventing bacterial or viral infection, or by reducing the risk of such an infection which would otherwise have prolonged the wound treatment process.

In another embodiment there is provided a method for treating damaged tissue in an individual, said method comprising the steps of contacting said damaged tissue with the wound or tissue dressing according to the invention, and treating the damaged tissue.

Likewise, the treatment can in principle result in healing of the damaged tissue or in accelerated healing of the damaged tissue. The accelerated healing can be a result of e.g. administration of a tissue-healing promoting substance, or a bioactive agent produced naturally or recombinantly by the lactic acid bacteria. Alternatively, the healing of damaged tissue can be promoted by preventing bacterial or viral infection, or by reducing the risk of such an infection which would otherwise have prolonged the treatment of the damaged tissue.

The tissue damage can e.g. be caused by bone protrudence, by diabetes, by circulatory insufficiencies or by undesirable inflammatory processes in an individual.

There is also provided a method for preventing or reducing the risk of wound or tissue infection in an individual having suffered a wound or damaged tissue, said method comprising the steps of contacting said wound or tissue with the wound or tissue dressing according to the invention, and treating the wound or tissue at risk of being infected. The infectious agent at risk of infecting the wound or tissue can be a pathogenic bacteria.

As e.g. gelatin and hyaluronic acid independently and in combination have a haemostatic effect, there is also provided a method for promoting haemostasis in a wound in an individual, said method comprising the steps of contacting said wound with the wound dressing according to the invention, and promoting haemostasis in the wound.

In addition to contacting a wound or damaged tissue with the wound or tissue dressing according to the invention, there is also provided combination methods wherein one or more wound or tissue healing-promoting substance(s) are administered simultaneously or sequentially in any order one or more at the same time as the wound or tissue to be treated is contacted with the wound or tissue dressing according to the invention. This may be of particular importance when treating slow-healing wounds, partial thickness wound, deep wounds and chronic wounds.

Accordingly, at least one wound healing-promoting substance selected from the group consisting of alpha-1 antitrypsin, SLPI (Secretory Leukocyte Protease Inhibitor), PDGF (Platelet Derived Growth Factor), rhPDGF-BB (Becaplermin), EGF (Epidermal Growth Factor), PDECGF (Platelet Derived Endothelial Cell Growth Factor), aFGF (Acidic Fibroplast Growth Factor), bFGF (Basic Fibroplast Growth Factor), TGF-alpha (Transforming Growth Faxtor alpha), TGF-beta (Transforming Growth Factor beta), KGF (Keratinocyte Growth Factor), VEGF (Vascular endothelial growth factor), and IGF1/IGF2 (Insulin-Like Growth Factor), can be administered simultaneously or sequentially in any order with the bioactive agent produced by the lactic acid bacteria of the absorbent compound.

In further embodiments the present invention is directed to the following uses:

Use of a lactic acid bacteria in the manufacture of a wound or tissue dressing for treating a wound or tissue or accelerating the healing of a wound or tissue in an individual.

Use of a lactic acid bacteria in the manufacture of an absorbent compound for use in a wound or tissue dressing for treating a wound or tissue or accelerating the healing of a wound or tissue in an individual.

Use of a lactic acid bacteria in the manufacture of a wound or tissue dressing for preventing or reducing the risk of wound or tissue infection in an individual having suffered a wound.

Use of a lactic acid bacteria in the manufacture of a wound or tissue dressing for promoting haemostasis in a wound in an individual.

### EXAMPLES

### Example 1

Lyophilized Lactobacillus GG are resuspended in saline to a final concentration of 10⁹/ml. Gelatine sponges (2×2 cm) are kneaded in 0.8 ml of the lactobacillus suspension or in 0.8 ml saline, and transferred to LB plates previously plated with 100 ul of an 1/10 diluted on culture of Staphylococcus aureus, Psuedomonas aeruginosa or Escherichia coli. The plates are incubated at 37° C. After 3 days a growth inhibitory (clear) zone is visible around the gelatin sponges kneaded in the lactobacillus suspension but not around the sponges kneaded in saline.

### Example 2

In a two compartment syringe the one compartment is filled with 10 ml hydrogel, sealed and subsequently autoclaved. After the sterilization process, the other compartment is filled with 10¹⁰ lyophilized lactic acid bacteria formulated with suitable low moisture exhibiants (carboxymethyl cellulose and maltodextrin) and prebiotica (trehalose). Staphylococcus aureus, Psuedomonas aeruginosa or Escherichia coli cultures are grown to in exponential growth and 100 ul of the cultures and plated on LB plates when the cultures have reached an OD₆₀₀ of 0.5. Wells are made in the agar plates and these wells are filled with a plain hydrogel or with the lactobacillus containing hydrogel using the two compartment syringe. The plates are incubated at 37° C. After 3 days a growth inhibitory (clear) zone is visible around the wells containing the hydrogel with lactobacillus. No inhibitor zone is visible around the well containing the plain hydrogel.

### Example 3

Lyophilized Lactobacillus GG are resuspended in saline to a final concentration of 10⁹/ml and 1 ml of this suspension is filled in a dialysis tubing with a molecular weight cut offs at 300 kDa allowing molecules up to 300 kDa to pass, but retaining the bacteria in the tube. In another tube 1 ml of saline is added. The tubes are placed on LB plates previously plated with 100 ul of an 1/10 diluted on culture of Staphylococcus aureus, Psuedomonas aeruginosa or Escherichia coli. The plates are incubated at 37° C. After 3 days a growth inhibitory (clear) zone is visible around the tubings containing the lactobacillus suspension but not around the tubings containing saline.

1. A wound or tissue dressing comprising lactic acid producing bacteria, said dressing comprising an absorbent compound for absorbing wound exudate, wherein the absorbent compound and the lactic acid producing bacteria are present in different compartments of the dressing, wherein the lactic acid producing bacteria, or their metabolites, can migrate from the compartment containing the lactic acid producing bacteria to the wound environment.

2-20. (canceled)

21. The wound or tissue dressing according to claim 1, wherein the absorbent compound comprises or consists of gelatin and/or collagen.

22-45. (canceled)

46. The wound or tissue dressing according to claim 1, wherein the wound or tissue dressing further comprises hyaluronic acid, or a derivative thereof.

47. The wound or tissue dressing according to claim 1, wherein the lactic acid bacteria is selected from the group consisting of Carnobacterium, Enterococcus, Lactobacillus, Lactococcus, Leuconostoc, Oenococcus, Pediococcus, Streptococcus, Tetragenococcus, Vagococcus and Weissella.

48. The wound or tissue dressing according to claim 1, wherein the lactic acid producing bacteria is present in said different compartment by physical entrapment.

49. The wound or tissue dressing according to claim 48 wherein said physical entrapment is within a matrix present in said different compartment.

50. The wound or tissue dressing according to claim 1, wherein the lactic acid producing bacteria is present in said different compartment by chemical bonding to a matrix.

51. The wound or tissue dressing according to claim 50 wherein said chemical bonding to a matrix is a gelatin and/or collagen matrix.

52-56. (canceled)

57. The wound or tissue dressing according to claim 1, wherein the absorbent compound is in contact with one or more compartment(s) comprising the lactic acid producing bacteria.

58. The wound or tissue dressing according to claim 57, wherein the compartment comprising the lactic acid producing bacteria is positioned on the proximal side of the absorbent compound between the absorbent compound and the wound.

59. (canceled)

60. The wound or tissue dressing according to claim 1, wherein the compartment comprising the lactic acid producing bacteria comprises different membrane portions, wherein each portion has permeable or semi-permeable characteristics with respect to the migration of the lactic acid producing bacteria and their metabolites.

61. The wound or tissue dressing according to claim 60, wherein one area or side of the compartment comprising the lactic acid producing bacteria comprises a membrane portion allowing migration of the lactic acid producing bacteria to the wound, whereas another side or area of the compartment comprising the lactic acid producing bacteria allows migration to the absorbent compound of metabolites only.

62-64. (canceled)

65. The wound or tissue dressing according to claim 1, wherein said dressing protected from undesirable contamination by an outer packaging comprising a sterile barrier seal in the form of an unbroken seal.

66. A method for manufacturing the wound or tissue dressing according to claim 1, said method comprising the steps of providing a lactic acid producing bacteria, attaching said lactic acid producing bacteria with the wound or tissue dressing in a compartment different from the absorbent compound of the wound or tissue dressing, thereby obtaining the wound or tissue dressing according to any of claim 1.

67-98. (canceled)

99. A method for manufacturing a wound or tissue dressing for treating a wound or tissue or accelerating the healing of a wound or tissue in an individual comprising the steps of:
i) providing a lactic acid bacteria,
ii) providing a wound or tissue dressing,
iii) incorporating said lactic acid bacteria into said wound or tissue dressing,
thereby obtaining a wound or tissue dressing for treating a wound or tissue or accelerating the healing of a wound or tissue in an individual.

100. A method for manufacturing an absorbent compound of a wound or tissue dressing for treating a wound or tissue or accelerating the healing of a wound or tissue in an individual comprising the steps of:
i) providing a lactic acid bacteria,
ii) providing an absorbent compound of a wound or tissue dressing,
iii) incorporating said lactic acid bacteria into said absorbent compound,
thereby obtaining an absorbent compound of a wound or tissue dressing for treating a wound or tissue or accelerating the healing of a wound or tissue in an individual.

101. A method for manufacturing a wound or tissue dressing for preventing or reducing the risk of wound or tissue infection in an individual having suffered a wound comprising the steps of:
i) providing a lactic acid bacteria,
ii) providing a wound or tissue dressing,
iii) incorporating said lactic acid bacteria into said wound or tissue dressing,
thereby obtaining a wound or tissue dressing for preventing or reducing the risk of wound or tissue infection in an individual having suffered a wound.

102. A method for manufacturing a wound or tissue dressing for promoting haemostasis in a wound in an individual comprising the steps of:
i) providing a lactic acid bacteria,
ii) providing a wound or tissue dressing,
iii) incorporating said lactic acid bacteria into said wound or tissue dressing,
thereby obtaining a wound or tissue dressing for promoting haemostasis in a wound in an individual.

103. A wound or tissue dressing comprising lactic acid producing bacteria, said dressing comprising an absorbent compound for absorbing wound exudate, wherein the absorbent compound and the lactic acid producing bacteria are present in different compartments of the dressing, wherein the lactic acid producing bacteria, or their metabolites, can migrate from the compartment containing the lactic acid producing bacteria to the wound environment, said wound or tissue dressing being for treating a wound or tissue or accelerating the healing of a wound or tissue in an individual, for preventing or reducing the risk of wound or tissue infection in an individual having suffered a wound, or for promoting haemostasis in a wound in an individual.
